(19) United States
(12) Patent Application Publication
(10) Pub. No.: US 2009/0270972 A1
(43) Pub. Date: Oct. 29, 2009

(54) ENDOVENOUS VALVE TRANSFER STENT

(75) Inventor: Rodney James Lane, New South Wales (AU)

Correspondence Address: PEARNE & GORDON LLP, 1801 EAST 9TH STREET, SUITE 1200, CLEVELAND, OH, 44114-3108, US

(73) Assignee: All-Vascular Pty Ltd., Castlecrag, New South Wales (AU)

(21) Appl. No.: 12/301,647

(22) PCT Filed: May 23, 2006
(86) PCT No.: PCT/AU06/00713

§ 371 Date: Dec. 30, 2008

(51) Int. Cl. A61F 2/06

(52) U.S. Cl. 623/1.14; 623/1.24

(57) ABSTRACT

An endovenous valve transfer stent (10) has an elongated tubular open-work body (12) having a network of longitudinally extending ribs (13) interconnected by laterally extending zigzag struts (14). The struts define a plurality of barbs (15, 17) in opposing longitudinal directions. The body (12) has a first end and a second end, and includes a longitudinal cut through the struts (14) from the first end to the second end. A seam (19) is defined between adjacent cut ends of each strut. The body (12) is adapted, in use, to vary in its diameter to receive a donor valve containing vein segment (20) longitudinally therethrough.

### FIELD OF INVENTION

This invention relates to surgical stents and more particularly to an endovenous valve transfer stent for transferring a donor valve containing vein segment to a recipient vein having a defective or absent venous valve. More particularly, the present invention relates to an endovenous valve transfer stent for treatment of chronic venous disease.

### BACKGROUND OF THE INVENTION

Chronic venous insufficiency imposes an enormous clinical and financial burden on the community with current treatment modalities being unsatisfactory. The syndrome relates to venous valve dysfunction leading to venous reflux, outflow congestion and venous hypertension. It is well known that this may lead to varicose veins, chronic venous ulcers and other related conditions in the long term.

Venous valves repaired directly or by venous valve transposition have produced encouraging results in the short and long term. However, these procedures require considerable surgical skill, and may be associated with potentially serious complications, specifically, deep venous thrombosis and pulmonary embolism, and so are not commonly performed. Additionally, there are problems related to valve ring dilatation and subsequent incompetence in the long term. A venous valve delivered intravenously and perhaps percutaneously may alleviate some of these logistical difficulties; and this has led to the development of artificial venous valves with promising results in non-human animals. However, an alternative approach that is likely to bring improvements over the use of artificial venous valves is the development of vascular stent technology to create an endovenous valve transfer stent.

### SUMMARY OF THE INVENTION

It is an object of the present invention to provide an endovenous valve transfer stent (EVTS) that may be used to treat chronic venous insufficiency in humans.

Further objects of the invention are to provide an exo-stent with a variable diameter i.e. placed circumferentially around the valve containing vein segment usually in the axilla or contralateral profunda vein. The anastomosis to the stent would need to be fluid sealed to prevent endoleak. The stent itself would need to have minimal blood interface to minimise thrombogenicity and, in order to prevent long term dilatation of the venous valve ring, the final stent diameter would need to be fixed to prevent long term dilatation.

Accordingly, the present invention provides an endovenous valve transfer stent comprising an elongated tubular open-work body having a network of longitudinally extending ribs interconnected by laterally extending zigzag struts that define a plurality of barbs in opposing longitudinal directions, the body having a first end and a second end, the body including a longitudinal cut through the struts from the first end to the second end so as to define a seam between adjacent cut ends of each strut, the body being adapted, in use, to vary in its diameter to receive a donor valve containing vein segment longitudinally therethrough.

Preferably, the ribs define outwardly projecting spikes at the first and second ends of the body which are adapted to secure the donor segment to the body of the stent.

It is preferred that the spikes have free ends which are adapted to penetrate opposite end walls of the donor segment.

In a preferred form, the stent is made of a nickel titanium alloy.

Preferably, the adjacent cut ends of each strut define hooks adapted to secure the donor segment to a recipient vein.

According to another aspect of the invention, there is provided a stented graft comprising the stent described above and a donor segment received longitudinally through the body and secured thereto by the spikes at the first and second ends of the body.

Preferably, the donor segment has an annular wall portion at an open end thereof that is outwardly folded back upon the stent and the spikes penetrate the folded back wall portion so as to secure the donor segment to the stent.

### BRIEF DESCRIPTION OF THE DRAWINGS

FIG. 1 is a side view of an endovenous valve transfer stent according to one embodiment of the present invention,

FIG. 2 is a schematic view of a vascular stent of the prior art installed in a vein across a tributary and showing perigraft haematoma causing extrinsic compression of the donor valve containing vein segment received in the stent,

FIG. 3 is a view similar to FIG. 2 showing an incompetent segment or endoleak in which retrograde flow bypasses the competent donor valve containing vein segment received in a vascular stent of the prior art,

FIG. 4 is a side view of the endovenous valve transfer stent shown in FIG. 1 and a donor valve containing vein segment intussuscepted over the spikes of the stent to form a stented graft according to another embodiment of the present invention, the stented graft being positioned within a recipient vein.

FIG. 5 is a side view of one stage in an intussusception securing process in which a similar stent to that of FIG. 1 has a donor segment secured thereto to form a stented graft by use of a dilator device.

FIG. 6 is a sectional view along a longitudinal plane of the arrangement shown in FIG. 5.

### MODES FOR CARRYING OUT THE INVENTION

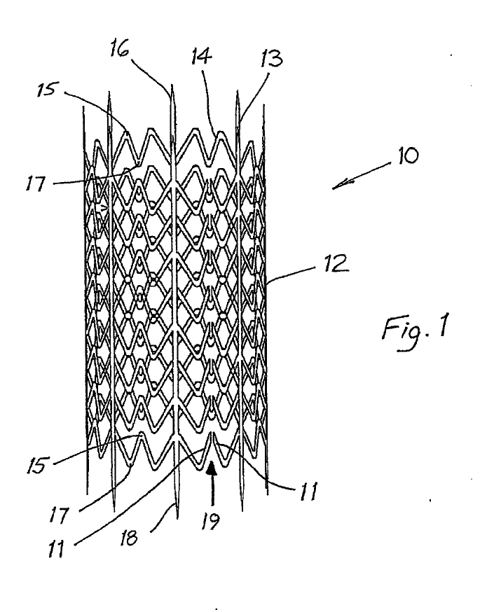

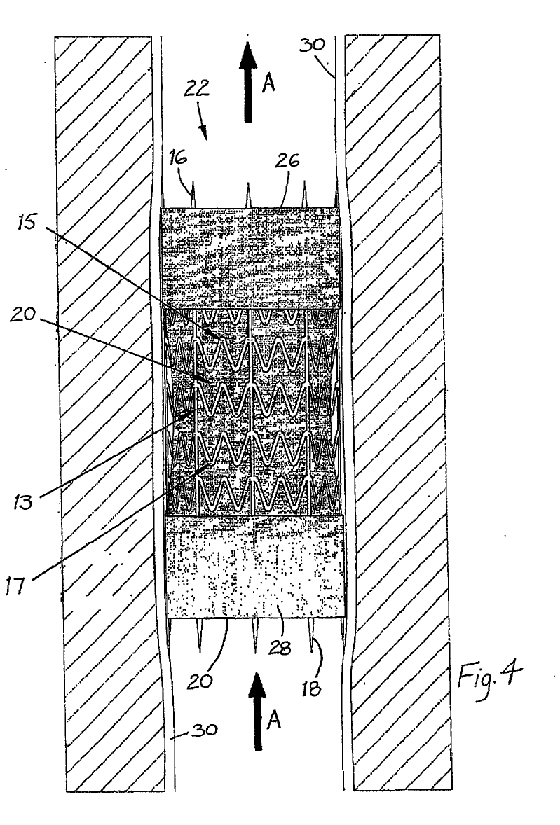

The endovenous valve transfer stent 10 shown in FIGS. 1 and 4 comprises an elongated tubular open-work body 12 having a network of longitudinally extending ribs 13 interconnected by laterally extending zigzag struts 14 that define a plurality of generally v-shaped barbs 15, 17 in opposing longitudinal directions.

The body 12 has a first end and a second end and is cut through the struts 14 longitudinally therealong. A seam 19 is defined where the struts 14 have been cut. The free cut ends of each strut 14 define hooks 11.

The body 12 has a wall defined by the ribs 13 and struts 14 that is less than or equal to about 250 μm in thickness.

At the opposed ends of the stent, the ribs define outwardly projecting spikes 16, 18, which optionally are barbed.

The stent is, in this embodiment, made of a nickel titanium alloy (referred to by the trade mark NITINOL) with elastic shape memory characteristics. Radio opaque markers (which may be metallic or polymeric) are present around each end to enable localization of the stent during an operation. The free ends of the spikes 16, 18 are sufficiently sharp to penetrate the wall of a donor valve containing vein segment to be carried by the stent. The free ends of the spikes 16, 18 are also adapted to be received by a recipient vessel when it is intended to anastomose a donor segment carried by the stent to an open end or other opening of the recipient vessel.

The stent 10 has a variable diameter arising from the longitudinal cut which forms the seam 19. Its elastic shape memory characteristics also contribute to allowing its diameter to be temporarily enlarged or contracted to receive a donor segment therein. That is, the diameter of the stent 10 can be varied to receive, or suit its circumferential placement around, a donor valve containing vein segment.

The structure of the body 12 may provide for varying radial force to be imposed along the length of the stent when it is located in a recipient vein.

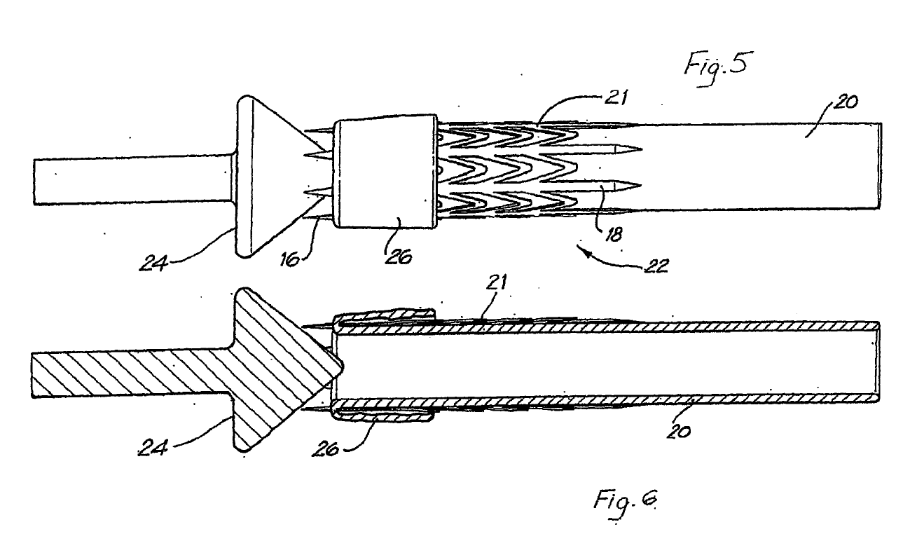

A donor valve containing vein segment 20, which may be of human or non-human animal origin, natural, artificial or engineered, is secured to the stent 10 to form a stented graft 22 (such as shown in FIG. 4) by the following process partly shown in FIGS. 5 and 6, but with reference to a similar stent 21. The stent 21 is expanded and the specially prepared donor segment 20 is positioned concentrically therewithin and intermediate its opposed open ends. A tapered head of a dilator device 24 is then inserted (tapered end first) into a first open end of the donor segment that extends from the stent 21, and the first open end of the donor segment is caused to stretch outwardly by relative movement of the open end against the tapered head. During this movement, the wall at the first open end of the donor segment folds back upon itself which allows the sharp ends of the spikes 16 to penetrate the wall, thus securing the donor segment to the stent at its first open end. This process is repeated at the second open end to form a stented graft similar to the stented graft 22.

The stented graft 22 shown in FIG. 4 illustrates two fold back wall portions 26,28 and the sharp ends of the spikes 16, 18 projecting through the luminal side of each wall portion to create opposed donor vein intussusception.

The stented graft 22 shown in FIG. 4 has been inserted into a recipient vein 30. The projecting sharp ends of the spikes 16, 18 penetrate and grip into the lumen of the recipient vein, and the fold back wall portions 26, 28 exert lateral pressure thereagainst to seal the donor segment 20 to the recipient vein 30.

The arrows A show the direction of blood flow (towards the right atrium of the heart).

The plurality of longitudinal barbs 15, 17 projecting in both proximal and distal directions create a series of external fixation points of the adventitia of the recipient vein 30 to the stent 10. The presence of a seam 19 in the stent 10 allows the creation of competence and the maintenance of competence after diameter fixation by suture. The plurality of hooks 11 on opposite sides of the seam 19 prevent movement of the stented graft 22, thus avoiding pulmonary embolus. Also the structure of the stent 10 does not allow any foreshortening or lengthening of the stented graft 22 which would, if it were allowed to occur, disrupt the anastomosis of both ends. It would be particularly disruptive of the function of the valve in the donor valve containing vein segment carried by the stent if the stented graft were to undergo a concertina—like contraction or an over stretching.

### Example 1

Subjects. Sixteen sheep weighing between forty-five to fifty kilograms were used in the study. The investigative protocol was approved by the Animal Ethics Committee of the Northern Sydney Central Coast Health Service.

Stent Materials. NITINOL (nickel titanium alloy) was chosen to take advantage of its super-elastic properties including its self-expansion capability and a very low mass expansion ratio. The lower profile allows minimisation of the delivery system. NITINOL has known and reproducible bio-compatibility. The fatigue deformity strength and electromagnetic profiles and stress strain characteristics are well documented and easy to test. NITINOL itself is easy to shape and it also has shape memory characteristics which allow crimping capability when cooled.

Stent Design. A preferred endovenous valve transfer stent (EVTS) is shown in FIG. 1.

1. To maximise wall fixation, the following EVTS structural features were included:
a. Spikes (3 mm in length) that would easily penetrate the walls of both donor and recipient veins are incorporated at both ends of the stent. These spikes provide an easy way to connect the ends of the donor valve containing vein segment to the stent.
b. The body of the stent has zigzag struts defining barbs, the points of which create high frictional resistance between the external surface of the stent and the wall of the recipient vein.
c. To facilitate variability of the diameter for different venous valves, the stent is, as seen from an end, in the form of an incomplete circle created by cutting the stent body longitudinally.
d. The resulting longitudinal cut edges provide hooks or further barbs that can impinge on the wall of the recipient vein and therefore prevent or at least minimise embolisation.
e. In the assessment of the diameter of the recipient vessel, a 2 mm expansion is considered optimal, i.e. if the donor segment requires a 10 mm diameter stent, an 8 mm diameter recipient vessel is selected.
f. In use, the stented graft is placed distally, i.e. theoretically, immediate pulmonary embolism would be blocked by the introducing system.
g. The external diameter of the stented graft can be increased by intussuscepting the donor segment over the spikes (as shown in FIG. 4).

2. The length of the EVTS shown in FIG. 1 is 20 mm to accommodate the following:
a. To suit large valves as well as smaller ones.
b. To avoid tilting within the recipient vein.
c. To minimise endoleak, i.e. an artificial passage between the stented graft and recipient vein.
d. In the clinical situation it is possible that the stented graft will cross a tributary and the length of 20 mm avoids a haemodynamic disturbance.

Pre-surgical Assessment. Two portable, battery powered duplex scanners Sonosite™ (Sonosite, Bothell, Wash.) and Terason 2000™ (Terason Ultrasound, Burlington, Mass.) were used to identify venous valves in the jugular systems. Completely competent or those with slightly incompetent valve segments were acceptable.

Surgical Procedure The sheep were anaesthetised with intravenous thiopentone following which they were intubated and ventilated. Both internal jugular veins were isolated and the valves identified. A NITINOL stent in accordance with the present invention was placed around the competent valve and the length of the vein segment fixed with sutures to prevent longitudinal shortening. Further 5-0 Prolene™ sutures were used to adjust the diameter of the EVTS and similarly sutures were used to anastomose the end of the EVTS to the donor valve containing vein segment. In this way the length of the segment is fixed to minimise the longitudinal contraction that occurs when the vein is divided. The cut ends of the vein were then formally anastomosed to the EVTS. This can also be achieved using mini-clips. The EVTS and the venous valve segment were then placed into the flared end of an introducing system (such as a modified 22 French introducing system) and a pusher is used to position the stented valve at the front end of the introducing system. After controlling the recipient vein with Vessiloops™ a venotomy allows deployment of the EVTS and valve segment.

Competence was tested by leaving the venotomy open. Absence of back flow when the proximal Vessiloop™ was released indicates competence. Post-operatively the sheep were returned to their pen and daily Clexane™ 40 mgs given subcutaneously for one week. The veins were then re-operated. The findings in one sheep acutely, and in five sheep each at one month, three months and six months (for a total of sixteen) were recorded.

End Points An Intra operative assessment and a post-operative assessment were made of patency, competence, thrombosis, tilting of the valve, migration, endoleak, fixation, stent visibility after venotomy, infection or other complications. Scanning E/M and light microscopy were performed on a total of seven specimens.

Results

Operative time taken=90 to 150 minutes

All sixteen specimens had no evidence of thrombosis, EVTS migration, tilting and no stents were visible. There were no endoleaks, although there were two cases where a tributary entered along the body of the stent. The tributaries remained open and there was no evidence of perigraft haematoma.

Microscopic

Microscopicly there was no evidence of thrombosis, cusp thickening or inflammatory changes or evidence of intimal hyperplasia or cellular infiltrate. The microscopic SEM findings showed no thrombosis cusp changes, intimal hyperplasia, cellular infiltrate, scanning, electro-microscopy, stent visibility, and cellular characteristics—i.e. normal structure.

### Discussion

These animal experiments demonstrate that an autologous venous valve mounted on a bio-compatible self-expanding stent can be introduced remotely with a patency of 100% with no loss of competence up to six months. This confirms similar experiments in goats and dogs using stents of the prior art. The surgical skill level required is minimal and many of the intrinsic problems with free venous valve transfer can be overcome with the use of the present invention. The potential advantages of use of the EVTS of the present invention over other procedures to correct or transplant deep venous valves include:
ring dilatation is prevented
recipient site can be selected
smaller valves can be harvested
multiple valves can be inserted by the same system.

The initial technical problems of spasm of the donor valve are obviated by applying the stent externally and varying its diameter. An alternative approach is to use various sized stents of the present invention, divide the donor vein distally and re-distend with an infusion cannula and syringe.

### Example 2

Case Report. A sixty-three year old man presented with a thirty-four year history of virtually continuous right lower limb chronic venous ulceration following an extensive DVT related to severe trauma. Treatment over the years consisted of continuous graduated compression, multiple failed skin grafts and a high ligation and stripping of the great saphenous vein plus other ablative venous procedures. He had multiple infective problems including multiple admissions to hospital for recurrent septicaemia. At the time of the EVTS procedure his ulcer area was 45 cm². A Venogram and duplex ultrasound of his left upper limb both demonstrated competent valves with internal diameters (ID) of 9 mm and 7 mm. Descending venography showed Grade IV reflux that extended from the femoral veins down to and including the infrapopliteal systems. Extensive post-phlebitic intraluminal changes were noted including vein wall thickening and irregularity. The popliteal vein ID varied between 8-10 mm.

In the pre-op workup, duplex ultrasound identified the site of the donor valves (with skin marking) and their size. The recipient site was also selected with help of the ascending and descending venograms and the duplex scanner. A site of appropriate size with smooth walls was optimal.

Under general anaesthetic the left axillary vein segment containing the valve was externally stented at 8.5 mm ID using a NITINOL stent of the present invention. The segment was anastomosed to the ends of the stent using 5-0 Prolene™ leaving a free segment of vein containing valve. This was tested and demonstrated to be competent by the “Milking” technique. The above knee popliteal vein was dissected and controlled with Vessiloops™ and through a small longitudinal incision the lower popliteal and tibial systems were dilated and the EVTS deployed proximal to the tibio peroneal trunk. The operative descending venogram demonstrated a patent and competent valve. Post operatively the duplex scan confirmed a patent competent popliteal valve. The patient sustained no complications including no arm swelling and was continued on Clexane™ for three days after which he was prescribed Warfarin™ for six weeks.

Ultrasound at three months postoperative confirmed the patency and competency of the transferred venous valve.

### Discussion

Various advantages are apparent from the following structural features of the endovenous valve transfer stent (EVTS) of the present invention:

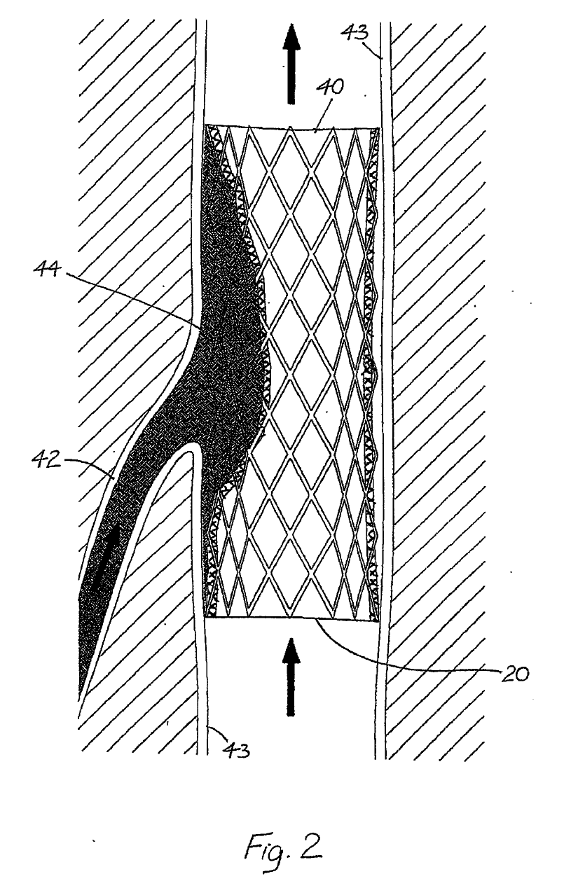

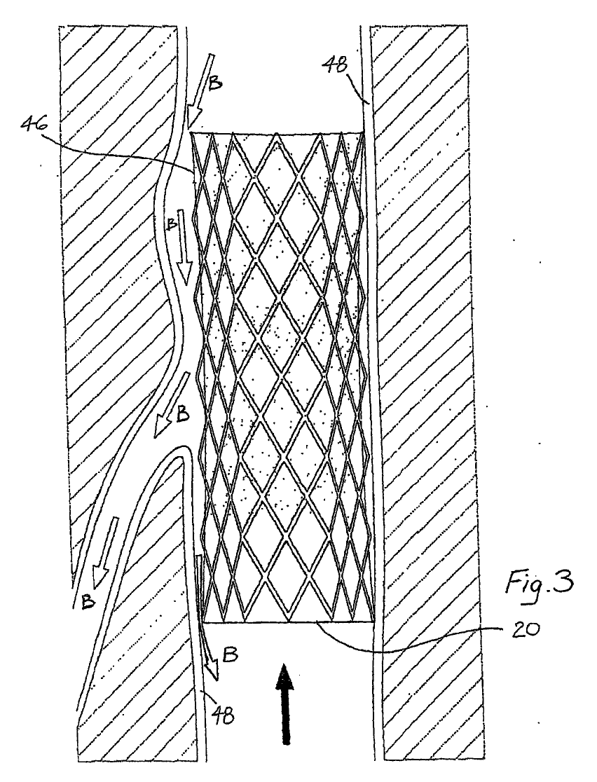

1. The longitudinal spikes at either end of the EVTS
(a) are long enough to penetrate the transferred section of vein (i.e. donor segment) and the recipient vein,
(b) prevent migration and embolisation of the EVTS,
(c) are in close proximity to each other around the circumference of the EVTS to increase attachment area and prevent migration and endoleak,
(d) allow the proximal and distal ends of the transferred section of vein to be cuffed over the respective ends of the EVTS to facilitate direct endothelial-endothelial contact (between the transferred and recipient veins) and therefore significantly reduce the risk of thrombosis.
   2. The barbs in opposing longitudinal directions of the body of the EVTS
(a) allow rapid adventitial adhesion of the transferred section of vein to the EVTS and to the recipient vein,
(b) aid in fixation of the transferred section of vein and the EVTS to the recipient vein, therefore preventing mobilisation and migration of the EVTS and subsequent pulmonary embolus,
(c) prevent detachment of the transferred section of vein and the collection of blood between the transferred section of vein and the recipient vein—known as a “perigrapft haematoma” (see FIG. 2),
(d) prevent detachment of the transferred section of vein from the recipient vein at either the proximal or distal end of the EVTS which could allow an endoleak (see FIG. 3).
   3. The length of the EVTS
(a) increases the surface area for attachment,
(b) prevents tilting of the transferred section of vein.
   4. Expansion of the EVTS (due to the properties of the NITINOL material) allows rapid and accurate deployment and attachment of the EVTS to the recipient vein, therefore reducing the risk of EVTS migration and embolisation.
   5. Design of the longitudinal ribs of the EVTS prevents changes in the length of the EVTS when the temperature or diameter of the EVTS is varied. This therefore prevents over stretching or foreshortening of the transferred section of vein which would lead to failure of the valve.
   6. The EVTS is cut longitudinally
(a) to allow the EVTS to be opened flat across a seam and passed around the section of vein to be transferred before that vein is cut, (this prevents twisting, shortening, or lengthening of the transferred section of vein),
(b) to provide hooks along the longitudinal cut edges of the EVTS so as to allow enhanced attachment of the EVTS to the recipient vein.

Non-endovenous valve transfer stents of the prior art have a variable longitudinal diameter which enables them to be collapsed easily and therefore be inserted into an introducing system. However collapsibility of endovenous valve transfer stents is not desirable. As shown in FIG. 2, if the stented graft 40 of the prior art crosses a tributary 42 of the recipient vein 43, then a perigraft haematoma 44 will form and obstruct the graft. Therefore, an important feature of the stent of the present invention (in order to avoid collapsing in situ) is the large number of barbs defined by the zigzag struts throughout the body of the stent. This feature also prevents a venous endoleak (see FIG. 3) which is an abnormal communication (or incomplete attachment) between the wall of the donor segment 46 and recipient vein 48. The arrows B show the direction of retrograde blood flow in the region of incomplete attachment.

Another advantage of the stent of the present invention is that it avoids foreshortening or over stretching of its length. If the length of the stent is not fixed, then any stretching of the stent will stretch the donor segment which may lead to deformity within the valve itself. Similarly if the length of the stent is shortened, then this may create a concertina-like effect within the donor segment and cause obstruction and therefore loss of valve function. Also, the abnormal shortening or lengthening described above may cause disruption of the anastomosis at either end.

The stent of the present invention is suited, not only to creating valve competence, but to transferring a competent vein segment in a donor to another area in the donor where valve competence is required. Some 25% of the valves in the arm for example are incompetent and therefore this stent is able to create competence as well as transfer it to a different area. More importantly, if the valve itself is simply transferred without a stent, then the native valve ring dilates and becomes incompetent and there is, therefore, recurrence of the chronic venous hypertension.

Yet another advantage of the stent of the present invention is that the hooks provided at adjacent cut ends of each strut forming the stent prevent movement of the stented graft, which would otherwise cause an embolism.

Various modifications may be made in details of design and construction without departing from the scope and ambit of the invention.

1. An endovenous valve transfer stent comprising an elongated tubular open-work body having a network of longitudinally extending ribs interconnected by laterally extending zigzag struts that define a plurality of barbs in opposing longitudinal directions, the body having a first end and a second end, the body including a longitudinal cut through the struts from the first end to the second end so as to define a seam between adjacent cut ends of each strut, the body being adapted, in use, to vary in its diameter to receive a donor valve containing vein segment longitudinally therethrough.

2. The stent of claim 1 wherein the ribs define outwardly projecting spikes at the first and second ends of the body which are adapted to secure the donor segment to the body of the stent.

3. The stent of claim 2 wherein the spikes have free ends which are adapted to penetrate opposite end walls of the donor segment.

4. The stent of claim 1 wherein the adjacent cut ends of each strut define hooks adapted to secure the donor segment to a recipient vein.

5. The stent of claim 1 wherein the barbs are generally v-shaped and are adapted to prevent movement and facilitate adhesion of the donor segment within the recipient vessel.

6. The stent of claim 1 wherein the stent is made of nickel titanium alloy.

7. The stent of claim 1 wherein the body has a tubular wall that is less than or equal to about 250 μm in thickness.

8. A stented graft comprising the stent of claim 1 and a donor segment received longitudinally through the body and secured thereto.

9. A stented graft comprising the stent of claim 3 and a donor segment received longitudinally through the body and secured thereto by the spikes at the first and second ends of the body.

10. The stented graft of claim 9 wherein the donor segment has an annular wall portion at an open end thereof that is outwardly folded back upon the stent and the spikes penetrate the fold back wall portion so as to secure the donor segment to the stent.
